SEQUENCE LISTING (27 sequences)

SEQ ID NO: 1 - RNA, 20 nt, synthetic construct
  source 1..20: mol_type = other RNA
tcttccttgccaagtgagaa

SEQ ID NO: 2 - DNA, 21 bp, synthetic construct
  source 1..21: mol_type = other DNA
cctgccctttcagttacttgg

SEQ ID NO: 3 - DNA, 24 bp, synthetic construct
  source 1..24: mol_type = other DNA
tgactaattagagagtggcccaga

SEQ ID NO: 4 - DNA, 24 bp, synthetic construct
  source 1..24: mol_type = other DNA
aaggttgatgaggaacagatgaaa

SEQ ID NO: 5 - DNA, 19 bp, synthetic construct
  source 1..19: mol_type = other DNA
ctcgtcatctctagcggca

SEQ ID NO: 6 - DNA, 19 bp, synthetic construct
  source 1..19: mol_type = other DNA
gccaagaagctgagaaccg

SEQ ID NO: 7 - DNA, 19 bp, synthetic construct
  source 1..19: mol_type = other DNA
ctcgaagctggccttcttc

SEQ ID NO: 8 - DNA, 77 bp, synthetic construct
  source 1..77: mol_type = other DNA
ttgcctttctcacttggcaaggaagatggaagtggggacagaggagatggcccctttcgcttaggtggccatgggcc

SEQ ID NO: 9 - DNA, 77 bp, synthetic construct
  source 1..77: mol_type = other DNA
aacggaaagagtgaaccgttccttctaccttcacccctgtctcctctaccggggaaagcgaatccaccggtacccgg

SEQ ID NO: 10 - protein, 26 aa, synthetic construct
LPFSLGKEDGSGDRGDGPFRLGGHGP

SEQ ID NO: 11 - DNA, 78 bp, synthetic construct
  source 1..78: mol_type = other DNA
ttgcctttcttaattggaaagggaaaatggaaggggggacggagaaattggcccttttcgctaagggggcctgggccc

SEQ ID NO: 12 - DNA, 63 bp, synthetic construct
  source 1..63: mol_type = other DNA
aagaagctgagaaccgaacaaggcggcgcccacttcagcgtgagctccctggccgagggctcc

SEQ ID NO: 13 - DNA, 63 bp, synthetic construct
  source 1..63: mol_type = other DNA
ttcttcgactcttggcttgttccgccgcgggtgaagtcgcactcgagggaccggctcccgagg

SEQ ID NO: 14 - protein, 21 aa, synthetic construct
KKLRTEQGGAHFSVSSLAEGS

SEQ ID NO: 15 - DNA, 63 bp, synthetic construct
  source 1..63: mol_type = other DNA
caggacaatcacgaggacggacctacagccaagaagctgagaaccgaacaaggcggcgcccac

SEQ ID NO: 16 - DNA, 63 bp, synthetic construct
  source 1..63: mol_type = other DNA
gtcctgttagtgctcctgcctggatgtcggttcttcgactcttggcttgttccgccgcgggtg

SEQ ID NO: 17 - protein, 21 aa, synthetic construct
QDNHEDGPTAKKLRTEQGGAH

SEQ ID NO: 18 - DNA, 78 bp, synthetic construct
  source 1..78: mol_type = other DNA
ttgcctttctcacttggcaaggaagatggaagtggggacagaggagatggcccctttcgcttaggtggccatgggcct

SEQ ID NO: 19 - DNA, 78 bp, synthetic construct
  source 1..78: mol_type = other DNA
aacggaaagagtgaaccgttccttctaccttcacccctgtctcctctaccggggaaagcgaatccaccggtacccgga

SEQ ID NO: 20 - protein, 65 aa, synthetic construct
TTGGCAAGGAAGATGGAAGTGGGGACAGAGGAGATGGCCCCTTTCGCTTAGGTGGCCATGGGCCT

SEQ ID NO: 21 - DNA, 54 bp, synthetic construct
  source 1..54: mol_type = other DNA
ctgttgatctttcttaatagcttgcctttctcacttggcaaggaagatggaagt

SEQ ID NO: 22 - DNA, 54 bp, synthetic construct
  source 1..54: mol_type = other DNA
gacaactagaaagaattatcgaacggaaagagtgaaccgttccttctaccttca

SEQ ID NO: 23 - protein, 11 aa, synthetic construct
LPFSLGKEDGS

SEQ ID NO: 24 - DNA, 41 bp, synthetic construct
  source 1..41: mol_type = other DNA
ctgttgatctttcttaatagcttggcaaggaagatggaagt

SEQ ID NO: 25 - DNA, 54 bp, synthetic construct; identical to SEQ ID NO: 21
  source 1..54: mol_type = other DNA

SEQ ID NO: 26 - DNA, 54 bp, synthetic construct; identical to SEQ ID NO: 22
  source 1..54: mol_type = other DNA

SEQ ID NO: 27 - DNA, 55 bp, synthetic construct
  source 1..55: mol_type = other DNA
ctgttgatctttcttaatagcttgcctttcttcacttggcaaggaagatggaagt

(19) United States
(12) Patent Application Publication
(10) Pub. No.: US 2026/0183428 A1
(43) Pub. Date: Jul. 2, 2026

(54) USE OF K568 SITE MUTATION IN Ku80 PROTEIN FOR REGULATING TUMOR CELL PROLIFERATION AND TUMOR CELL RADIOSENSITIVITY

(71) Applicant: Academy of Military Medical Sciences, AMMS, Beijing (CN)

(72) Inventors: Pingkun ZHOU, Beijing (CN); Shanshan GAO, Beijing (CN); Hua GUAN, Beijing (CN); Hongling ZHAO, Beijing (CN)

(21) Appl. No.: 19/416,054

(22) Filed: Dec. 11, 2025

(30) Foreign Application Priority Data
Dec. 31, 2024 (CN) 202411975547.3

(51) Int. Cl. A61K 48/00; C12N 15/86

(52) U.S. Cl. CPC A61K 48/0058; C12N 15/86; C12N 2740/15043

(57) ABSTRACT

Use of a K568 site mutation in a Ku80 protein for regulating tumor cell proliferation and tumor cell radiosensitivity, belonging to the technical field of biotechnology. Use of a substance capable of inducing a site-directed mutation at a K568 site of a Ku80 protein in an organism or a cell in any one of the following items: preparing a product for inhibiting proliferation of a tumor cell; preparing a product for inhibiting DNA damage repair in the tumor cell; preparing a product for treating a tumor; preparing a product for enhancing radiosensitivity of the tumor; and preparing a chemotherapeutic drug for tumor. Experimental results demonstrate that the K568 site mutation in the Ku80 protein reduces tumor cell viability, inhibits tumor cell proliferation, and promotes tumor cell apoptosis. Such a site also suppresses DNA damage repair in the tumor cell following radiotherapy, thereby enhancing radiosensitivity.

### CROSS REFERENCE TO RELATED APPLICATION

This patent application claims the benefit of and priority to Chinese Patent Application No. 202411975547.3, filed with the China National Intellectual Property Administration on Dec. 31, 2024, the disclosure of which is incorporated by reference herein in its entirety as part of the present application for all purposes.

### REFERENCE TO SEQUENCE LISTING

A computer readable XML file entitled “GWP20250701709.xml”, that was created on Oct. 30, 2025, with a file size of about 25,115 bytes, contains the sequence listing for this application, has been filed with this application, and is hereby incorporated by reference in its entirety.

### TECHNICAL FIELD

The present disclosure belongs to the technical field of biotechnology, and specifically relates to use of a K568 site mutation in a Ku80 protein for regulating tumor cell proliferation and tumor cell radiosensitivity.

### BACKGROUND

The Ku80 protein is a functionally significant protein within cells, typically forming a heterodimer with the Ku70 protein to constitute the Ku protein. The Ku protein possesses DNA recognition and binding capacity and modulates immune responses, both of which are implicated in tumorigenesis and tumor progression. Ku80 protein may additionally participate in other cellular physiological processes, such as cell cycle regulation and maintenance of chromosomal stability. However, the precise mechanisms underlying these functions remain incompletely elucidated.

Tumor represents one of the most severe diseases threatening human health. Tumorigenesis and tumor progression involve dysregulation of numerous intracellular molecular mechanisms. Current tumor therapeutics continue to face significant challenges, including acquired drug resistance in tumor cells during treatment and adverse side effects of therapeutic interventions on normal cells. Existing tumor treatment modalities exhibit substantial room for improvement in both efficacy and specificity. Consequently, exploring novel Ku80 protein-based tumor treatment strategies holds considerable scientific significance and promising clinical application prospects.

### SUMMARY

In order to solve the problems existing in the prior art, an objective of the present disclosure is to provide use of a K568 site mutation in a Ku80 protein for regulating tumor cell proliferation and tumor cell radiosensitivity.

To achieve the above objective, the present disclosure provides the following technical solutions:

The present disclosure provides use of a substance capable of inducing a site-directed mutation at a K568 site of a Ku80 protein in an organism or a cell in any one of the following items:
(1) preparing product for inhibiting proliferation of a tumor cell;
(2) preparing a product for inhibiting DNA damage repair in the tumor cell;
(3) preparing a product for treating a tumor;
(4) preparing a product for enhancing radiosensitivity of the tumor; and
(5) preparing a chemotherapeutic drug for tumor.

In some embodiments, the site-directed mutation at the K568 site is a substitution of K at position 568 of the Ku80 protein with R.

In some embodiments, the substance is any one or more selected from the group consisting of an shRNA, an siRNA, a dsRNA, an miRNA, a cDNA, an antisense RNA or an antisense DNA, a small-molecule compound, a peptide, and an antibody.

The present disclosure further provides use of a K568 site of a Ku80 protein in an organism or a cell as a target in developing any one of the following products:
(1) preparing a product for inhibiting proliferation of a tumor cell;
(2) preparing a product for inhibiting DNA damage repair in the tumor cell;
(3) preparing a product for treating a tumor;
(4) preparing a product for enhancing radiosensitivity of the tumor; and
(5) preparing a chemotherapeutic drug for tumor.

In some embodiments, the tumor is a Ku80 protein-expressing tumor, and the tumor cell is a Ku80 protein-expressing tumor cell.

The present disclosure further provides a method for inhibiting proliferation of a tumor cell for a non-diagnostic or non-therapeutic purpose, including: conducting a site-directed mutation at a K568 site of a Ku80 protein in the tumor cell.

The present disclosure further provides a method for inhibiting DNA damage repair in a tumor cell for a non-diagnostic or non-therapeutic purpose, including: conducting a site-directed mutation at a K568 site of a Ku80 protein in the tumor cell.

The present disclosure further provides a method for enhancing radiosensitivity of a tumor cell for a non-diagnostic or non-therapeutic purpose, including: conducting a site-directed mutation at a K568 site of a Ku80 protein in the tumor cell.

In some embodiments, the site-directed mutation at the K568 site is a substitution of K at position 568 of the Ku80 protein with R.

In some embodiments, the tumor cell is a Ku80 protein-expressing tumor cell.

Compared with the prior art, the technical solutions of the present disclosure have the following beneficial effects:

Experimental evidence confirms that the substitution of K with R at position 568 of the Ku80 protein reduces tumor cell viability, inhibits tumor cell proliferation, promotes tumor cell apoptosis, and suppresses DNA damage repair in the tumor cell following radiotherapy, thereby enhancing radiosensitivity. It further serves as a target for developing tumor-related drugs, showing a significant potential for clinical application.

### BRIEF DESCRIPTION OF THE DRAWINGS

FIGS. 1A-1B show sequencing results of Ku80 (K568R)-GFP-Flag DNA products;

FIG. 2 show Western blot detection of knockout efficiency and exogenous expression levels of target protein Ku80 in cells;

FIGS. 3A-3B show sequencing results of Ku80 (K568R)-GFP-Flag positive cells;

FIG. 4 shows comparison of sequencing results between PCR products from Ku80 (K568R)-GFP-Flag positive cells and the original sequence;

FIG. 5 shows Western blot detection of knockout efficiency and exogenous expression levels of target protein Ku80;

FIG. 6 shows comparison of sequencing results between PCR products from Ku80 (WT)-GFP-Flag positive cells and the original sequence;

FIG. 7 shows Western blot detection of knockout efficiency and exogenous expression levels of target protein Ku80 in Ku80 (WT)-GFP-Flag positive cells;

FIG. 8 shows Western blot detection of knockout efficiency and exogenous expression levels of target protein Ku80 in normal HELA cells, GFP-Flag-Ku80 (K568R) cell line, and GFP-Flag-Ku80 (WT) cell line;

FIG. 9 shows the effect of Ku80 K568R mutation on cell survival rate;

FIG. 10 shows the effect of Ku80 K568R mutation on cell growth;

FIG. 11 shows the effect of Ku80 K568R mutation on cellular response to different drug treatments;

FIGS. 12A-12B show the effect of Ku80 K568R mutation on change of γ-H2AX foci in cells;

FIGS. 13A-13B show the effect of Ku80 K568R mutation on cell apoptosis; and

FIGS. 14A-14C show the effect of Ku80 K568R mutation on tumor volume and weight in nude mice.

### DETAILED DESCRIPTION OF THE EMBODIMENTS

The present disclosure provides use of a substance capable of inducing a site-directed mutation at a K568 site of a Ku80 protein in an organism or a cell in any one of the following items:
(1) preparing a product for inhibiting proliferation of a tumor cell;
(2) preparing a product for inhibiting DNA damage repair in the tumor cell;
(3) preparing a product for treating a tumor;
(4) preparing a product for enhancing radiosensitivity of the tumor; and
(5) preparing a chemotherapeutic drug for tumor.

In some embodiments of the present disclosure, the inhibition of DNA damage repair in the tumor cell refers to enhancing DNA damage sensitivity in tumor cells; in some other embodiments, the inhibition of DNA damage repair in the tumor cell refers to enhancing DNA damage sensitivity in tumor cells post-irradiation while inhibiting DNA damage repair in tumor cells; in some embodiments, the irradiation is conducted using ⁶⁰Co γ ray irradiation.

In the present disclosure, the tumor is a Ku80 protein-expressing tumor; in some embodiments, the tumor is cervical carcinoma or breast cancer. The tumor cell is a Ku80 protein-expressing tumor cell; in some embodiments, the tumor cell is a cervical cancer cell or a breast cancer cell.

In the present disclosure, the site-directed mutation at the K568 site includes substitution of K (Lysine, Lys) at position 568 of the Ku80 protein with R (Arginine, Arg). This site-directed mutation will not cause frameshift mutant, meaning that the substance induces substitution of K at position 568 of the Ku80 protein with R in organisms or cells while other amino acid residues of the Ku80 protein remain unchanged.

In the present disclosure, the substance is any one or more selected from the group consisting of an shRNA, an siRNA, a dsRNA, an miRNA, a cDNA, an antisense RNA or an antisense DNA, a small-molecule compound, a peptide, and an antibody, but is not limited thereto.

The present disclosure further provides use of a K568 site of a Ku80 protein in an organism or a cell as a target in developing any one of the following products:
(1) preparing a product for inhibiting proliferation of a tumor cell;
(2) preparing a product for inhibiting DNA damage repair in the tumor cell;
(3) preparing a product for treating a tumor;
(4) preparing a product for enhancing radiosensitivity of the tumor; and
(5) preparing a chemotherapeutic drug for tumor.

In some embodiments of the present disclosure, the inhibition of DNA damage repair in the tumor cell refers to enhancing DNA damage sensitivity in tumor cells. In some embodiments, the inhibition of DNA damage repair in the tumor cell refers to enhancing DNA damage sensitivity in tumor cells post-irradiation while inhibiting DNA damage repair; and in some embodiments, the irradiation is conducted using ⁶⁰Co γ ray irradiation.

In the present disclosure, the tumor is a Ku80 protein-expressing tumor; in some embodiments, the tumor is cervical carcinoma or breast cancer. The tumor cell is a Ku80 protein-expressing tumor cell; in some embodiments, the tumor cell is a cervical cancer cell or a breast cancer cell.

In the present disclosure, the site-directed mutation at the K568 site is a substitution of K at position 568 of the Ku80 protein with R. This site-directed mutation will not cause frameshift mutant, meaning that the substance induces substitution of K at position 568 of the Ku80 protein with R in organisms or cells, while other amino acid residues of the Ku80 protein remain unchanged.

The present disclosure further provides a method for inhibiting proliferation of a tumor cell, suppressing DNA damage repair in the tumor cell, or enhancing radiosensitivity of the tumor cell, including: conducting a site-directed mutation at a K568 site of a Ku80 protein in the tumor cell.

In the present disclosure, the site-directed mutation at the K568 site is a substitution of K at position 568 of the Ku80 protein with R. This site-directed mutation will not cause frameshift mutant, meaning that the substance induces substitution of K at position 568 of the Ku80 protein with R in organisms or cells, while other amino acid residues of the Ku80 protein remain unchanged. In the present disclosure, the tumor cell is a Ku80 protein-expressing tumor cell, and in some embodiments, the tumor cell is a cervical carcinoma or breast cancer cell.

In the present disclosure, the method is for a non-diagnostic or non-therapeutic purpose. As a non-diagnostic or non-therapeutic method, it may be used for basic research on tumor cell proliferation or serve as a positive control in screening drugs capable of inhibiting tumor cell proliferation.

The technical solutions of the present disclosure will be clearly and completely described below with reference to the examples of the present disclosure. Apparently, the described examples are merely a part rather than all of the examples of the present disclosure. All other examples obtained by those skilled in the art based on the examples of the present disclosure without creative efforts shall fall within the protection scope of the present disclosure.

In the embodiments of the present disclosure, nude mice are purchased from SPF Biotechnology Co., Ltd. (Animal Production License No.: SCXK (Jing) 2019-0010); Lenti-HG Mix is obtained from Genomeditech/GMLCP (1 mg/mL).

In specific embodiments, the GFP-Flag-Ku80 (K568R) and GFP-Flag-Ku80 (WT) cell lines are constructed by Genomeditech (Shanghai) Co., Ltd. using the following methodology:

(1) CRISPR/Cas9 Vector Construction: single-stranded DNA oligos for the gRNA sequence (TCTTCCTTGCCAAGTGAGAA, SEQ ID NO: 1, denoted as Human XRCC5) are synthesized, annealed to form double-stranded DNA oligos, and ligated into the restriction enzyme-digested CRISPR/Cas9 vector (GM-31871: LentiGuide-CMV-Neo (stuffer), purchased from Genomeditech Co., Ltd.) via restriction sites contained at both ends. The ligation product is transformed into competent bacterial cells. Monoclonal colonies are sequenced, and clones with correct alignment yielded the successfully conducted H_XRCC5 sgRNA-Neo (lenti-SpCas9) plasmid.
(2) H_XRCC5 sgRNA-Neo Lentiviral Packaging:

293T cells at 70% to 80% confluency are co-transfected using: 850 μL DMEM, 10 μg plasmid, 10 μL Lenti-HG Mix (10 μg), and 60 μg HG transgene reagent. After 20 minutes (min) at room temperature, the mixture is added dropwise to culture dishes for transfection and incubated in a CO₂ incubator. After 12 hours (h) of transfection, 100×Enhancing buffer is added dropwise evenly to enhance transfection efficiency. After 20 h of transfection, medium is aspirated into disinfectant-containing waste vessels. Then 12 mL of DMEM cell medium containing 1% serum are added to continue the culture. After 48 h of medium change, supernatant is transferred into a 50 mL centrifuge tube, centrifuged at 4° C., 4,000 g for 5 min. The supernatant is filtered through 0.22 μm membranes and transferred to a new centrifuge tube. The filtrate is then aliquoted into a concentration device and concentrated at 4° C., 3,500 g for 10 min. The lower layer of liquid is discarded into a.disinfectant-containing waste vessels. A final centrifugation step is performed at 4° C., 3,500 g for 20 min. The liquid remaining in the upper chamber of the concentrator is the concentrated viral solution.

### (3) H_XRCC5 Overexpression Lentivirus Construction:

According to the mutation requirements of the K568R site of a Human XRCC5 gene, codon-optimized target gene/point mutation sequences are constructed into the GM-8077: PGMLV-CMV-MCS-eGFP-3×Flag-PGK-Puro vector (purchased from Genomeditech Co., Ltd.). Primers are designed to amplify the fragment, which is ligated into the restriction enzyme-digested vector. The ligation products are transferred into the prepared bacterial competent cells, and grown monoclonal colonies are sent to a sequencing company for sequencing. Clones with correct sequences, as confirmed by alignment, ARE considered successfully constructed vectors. This results in the generation of the overexpression plasmids PGMLV-CMV-H_XRCC5 (p.K568R) (Codon opt)-eGFP-3×Flag-PGK-Puro and PGMLV-CMV-H XRCC5 (Codon opt)-Egfp-3×Flag-PGK-Puro.

Lentiviral packaging is the same as described in step (2). Constructed lentiviral vector and its helper packaging element vector plasmid are co-transfected into 293T cells using HG transgene reagent. Enhancing buffer is added at 10 h to 12 h after transfection, and then fresh medium is replaced 8 h later. After continued culture for 48 h, supernatant rich in lentiviral particles is collected, concentrated, and high-titer lentiviral concentrate was obtained. The viral titer was determined and calibrated in 293T cells.

### (4) Cas9-Blast HELA Cell Construction:

HELA cells are infected with lenti Cas9-Blasticidin lentivirus (GM-0220LV03, purchased from Genomeditech Co., Ltd.) to obtain Cas9-Blast HELA stably-transformed cell line.

The experimental method is as follows: on the first day, 5E4 HELA cells/well are inoculated in 24-well plates. Before infection, virus stock solution from −80° C. refrigerator is thawed on ice, diluted in 500 μL complete medium (MOI=100), and the original culture medium in the treatment group is sucked off. 300 μL culture medium containing diluted lentivirus solution is added to the cells in the treatment group. Cuture medium replacement is conducted after 16 h infection by replacing culture medium containing diluted lentivirus solution with 500 μL complete medium. Appropriate resistance screening cells are selected (Blasticidin full lethal concentration 30 μg/mL, maintenance concentration 15 μg/mL), and two rounds of drug screening are conducted (1 round 3 d) until the cells are stable. After stabilization, DMEM+10% FBS+1% Pen/Strep+15 μg/mL Blasticidin complete medium is added to maintain the cells.

### (5) Construction of Ku80 (K568R)-GFP-Flag and Ku80 (WT)-GFP-Flag Cell Lines:

Cas9-Blast HELA cells are co-infected with pakaged H_XRCC5 (p.K568R) (Codon opt) and H_XRCC5 sgRNA6 lentivirus at a 1:1 ratio or H_XRCC5 (Codon opt) lentivirus and H_XRCC5 sgRNA6 lentivirus at a 1:1 ratio, yielding Ku80 (K568R)-GFP-Flag cell lines and

Ku80 (WT)-GFP-Flag cell lines.

The experimental method is as follows: 1E5 Cas9-blast HELA cells/well are inoculated in 12-well plates. Before infection, virus stock solution from −80° C. refrigerator is thawed on ice, diluted in 800 μL complete medium (MOI=100), and the original culture medium in the treatment group is sucked off. 500 μL of culture medium containing diluted lentivirus solution is added to the cells in the treatment group. Cuture medium replacement is conducted after 16 h infection by replacing culture medium containing diluted lentivirus solution with 1 mL complete medium. Detection of Infection efficiency is conducted by observing fluorescence under an inverted fluorescence microscope to estimate the efficiency of lentivirus infection of the target cells. Appropriate resistance screening cells are selected (Puromycin full lethal concentration: 1 μg/mL; maintenance: 0.5 μg/mL) and two rounds of drug screening are conducted until the cells are stable. After stabilization, DMEM+10% FBS+1% P.S+0.5 μg/mL Puromycin complete medium is added to maintain the cells.

### (6) Pooled Clone Sequencing and Western Blot Validation:

DNA is extracted using Beyotime DNA extraction kit, and the the specific procedures are performed according to the kit instructions The PCR amplification primers pre-designed for the H XRCC5 codon optimization sequence are synthesized for PCR, including: 56939CF2 (CCTGCCCTTTCAGTTACTTGG, SEQ ID NO: 2), 56939CR2 (TGACTAATTAGAGAGTGGCCCAGA, SEQ ID NO: 3), 75400CF1 (AAGGTTGATGAGGAACAGATGAAA, SEQ ID NO: 4), 75400CR1 (CTCGTCATCTCTAGCGGCA, SEQ ID NO: 5), 71713CF1 (GCCAAGAAGCTGAGAACCG, SEQ ID NO: 6), and 71713CR1 (CTCGAAGCTGGCCTTCTTC, SEQ ID NO: 7).

PCR amplification system: 100 ng template, 2 μL Test-F, 2 μL Test-R, 25 L PCR mix, and ddH₂O to 50 μL.

The above materials are added to a thin-walled tube, mixed thoroughly, centrifuged briefly, and then placed in a PCR instrument. Appropriate annealing and extension temperatures are selected to initiate PCR application. Post-PCR, agarose gel electrophoresis is conducted to recover the target gene. The recovered PCR products are sent for sequencing.

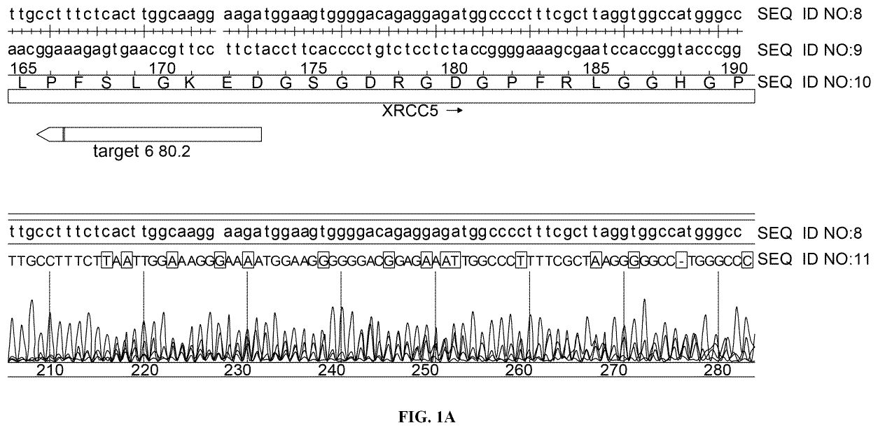

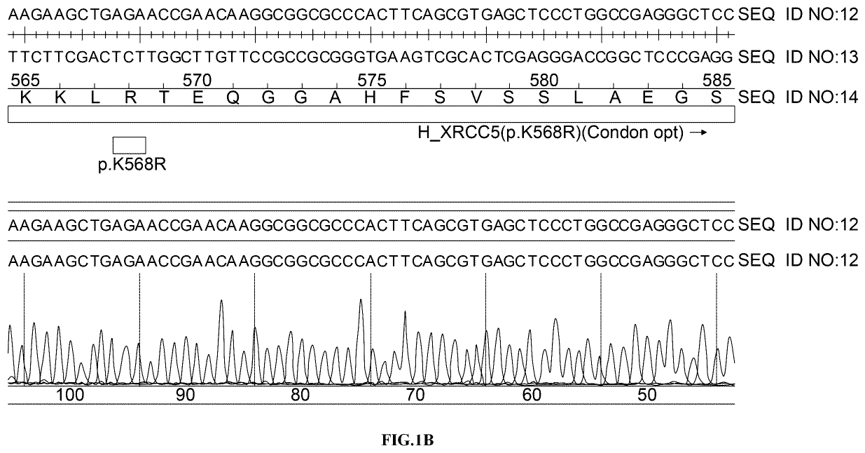

Sequencing results of Ku80 (WT)-GFP-Flag DNA products are not shown. The sequencing files are named: 0719_32723051603212_(75400-A1-3) [56939CF₂-75541]; 0194 32723052100165_(75400-A4-1A)_[75400CF1-86314]; 0195_32723052100165_(75400-A4-1A) _[75400CR1-86315]. Sequencing results of Ku80 (K568R)-GFP-Flag DNA products are shown in FIGS. 1A-1B. The sequencing files are named: 0695_32723051603200_(71713-A1-2) [56939CF₂-75541]; 0696 32723051603200_(71713-A1-2)_[56939CR2-75542]; 0253 32723051800716_(71713-A1-3)_[71713CF1-86305]; 0254 32723051800716_(71713-A1-3)_[71713CR1-86313].

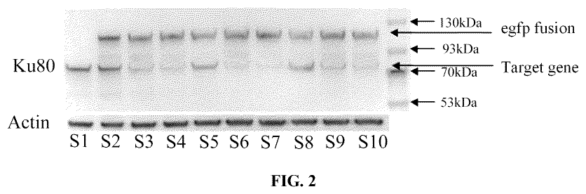

FIG. 2 shows Western blot detection of knockout efficiency and exogenous expression levels of target protein Ku80 in cells. Lane S2 represents HELA group that overexpressed the H_XRCC5 plasmid, S3 represents (Ku80 (WT)-GFP-Flag stably transformed plant group, S6 represents Ku80 (K568R)-GFP-Flag group (the rest lanes are other experimental results at the same group, which are reserved because they cannot be removed).

### (7) Monoclonal Colonies Isolation via Limited Dilution:

Stably transformed plant of Ku80 (K568R)-GFP-Flag and Ku80 (WT)-GFP-Flag are diluted in DMEM medium containing 20% FBS, 1% Pen/Strep, and 0.5 μg/mL Puromycin. After culturing 2-3 d, wells containing positive monoclonal colonies are marked. One week later, the growth of monoclonal colonies are observed and medium is replenished in a timely manner, After approximately three weeks, monoclonal colonies are transferred to 48-well plates for expansion. Cells reaching quantity suitable for 24-well plate are harvested for downstream assays.

### (8) Monoclonal Sequencing, TA Cloning and WB Validation:

DNA is extracted using a DNA extraction kit. PCR amplification is conducted using gRNA-specific primers and followed by gel extraction and sequencing for validation. For positive clones exhibiting different mutation patterns between the two alleles, TA cloning was repeated. The resulting clones were sent for sequencing and compared with the wild-type sequence to determine the mutation status of each allele.

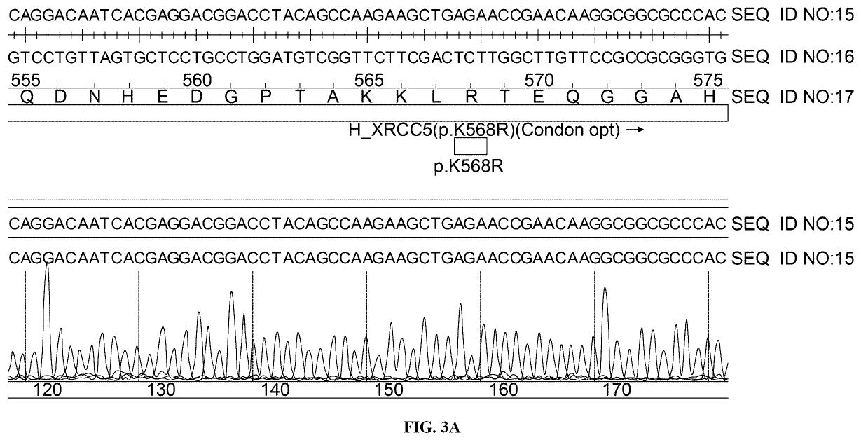

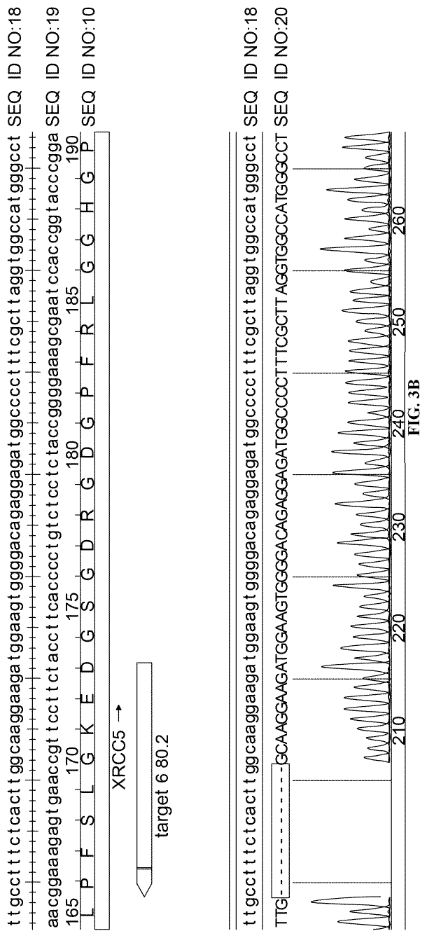

Based on PCR product sequencing results, cell #4 of Ku80 (K568R)-GFP-Flag is identified as positive, with sequencing results shown in FIGS. 3A-3B (71713D-A1-primer-71715CF1-88843_H03; 0005 32723061300656_(71713H-B5)_[56939CF₂-75541]).

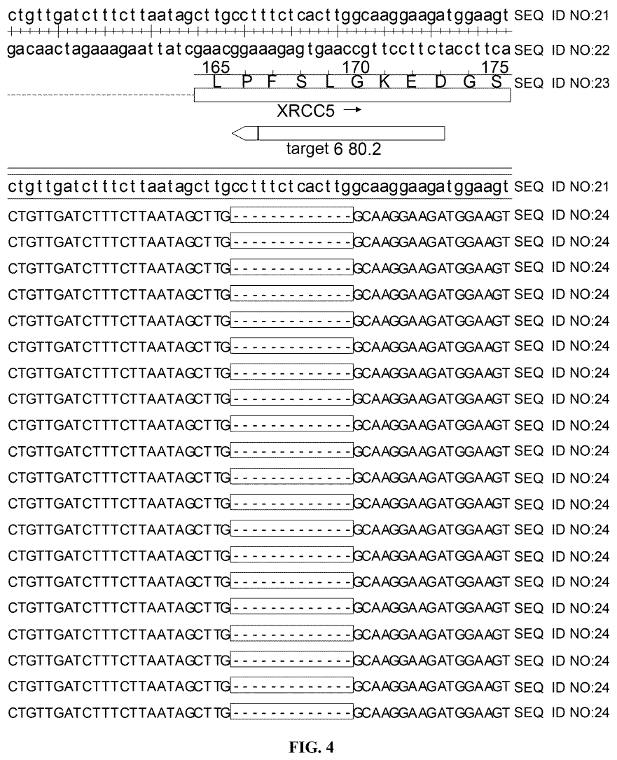

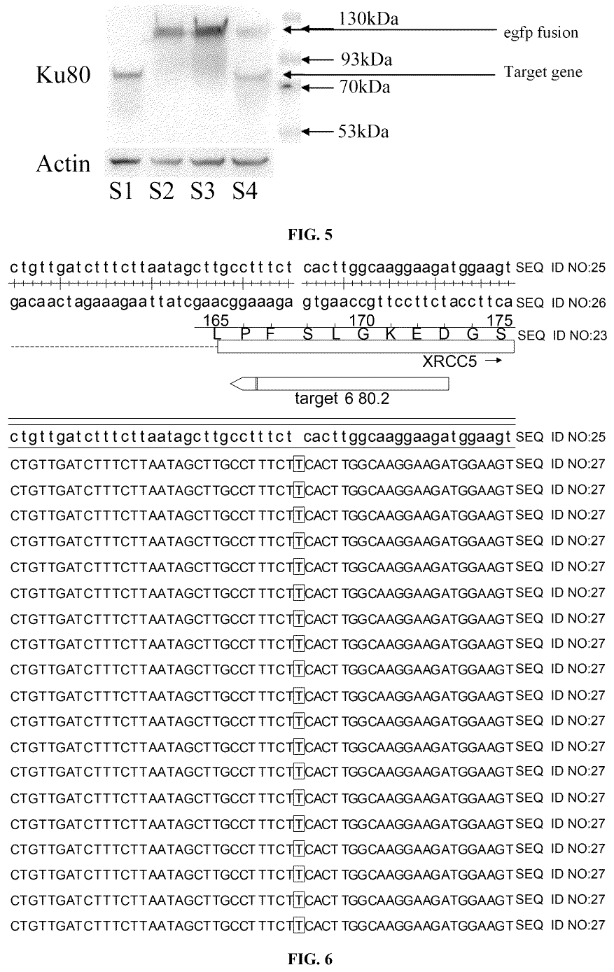

Comparison between PCR product sequencing results of #4 and the original sequence reveals exactly 1 variant type (excluding vector), and the specific sequencing results are detailed in FIG. 4. This clone exhibits a 13-bp deletion relative to the parental sequence, causing premature translational termination (including missense mutations). Western blot analysis of knockout efficiency and exogenous expression of Ku80 target protein in Ku80 (K568R)-GFP-Flag positive cells is presented in FIG. 5, where Lane S2 displays the verification result of Ku80 (K568R)-GFP-Flag (other lanes represent other experimental results from the same group, which are reserved because they cannot be removed).

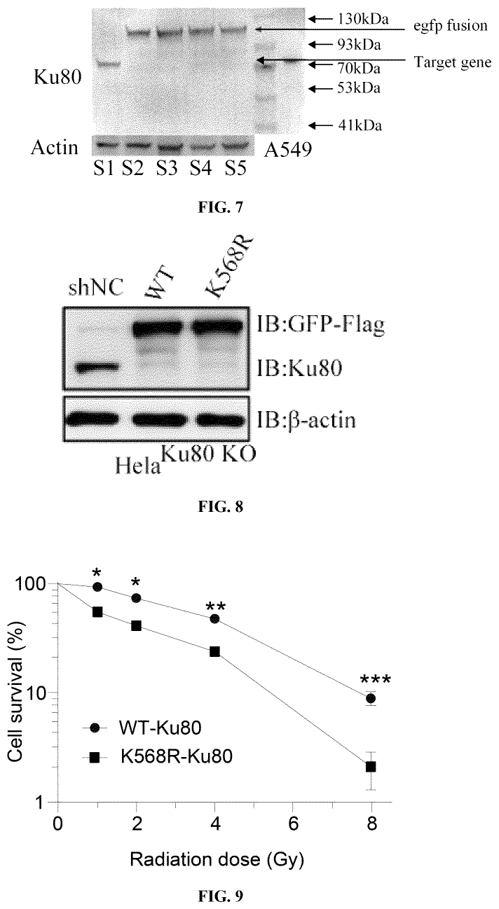

Based on PCR product sequencing results, cell #24 of Ku80 (WT)-GFP-Flag is identified as positive, but the sequencing results are not shown (75400MY-2C-primer-75400CF1A-95789_C04; 75400MY-2C-primer-75400CR1A-95790_D04; 56940CD-D7-primer-56939CF₂-75541_D07). Comparison between PCR product sequencing results of #24 and the original sequence reveals exactly 1 variant type (excluding vector), and the specific sequencing results are detailed in FIG. 6. This clone exhibits a 1-bp insertion relative to the parental sequence, causing premature translational termination (including missense mutations). Western blot analysis of knockout efficiency and exogenous expression of Ku80 target protein in Ku80 (WT)-GFP-Flag positive cells is presented in FIG. 7, where Lane S4 displays the verification result of Ku80 (WT)-GFP-Flag (other lanes represent other experimental results from the same group, which are reserved because they cannot be removed).

In FIG. 8, shNC denotes the normal HELA cell line; K568R and WT represent the GFP-Flag-Ku80 (K568R) and GFP-Flag-Ku80 (WT) cell lines, respectively.

Collectively, these results demonstrate that: compared to the shNC cell line, both GFP-Flag-Ku80 (K568R) and GFP-Flag-Ku80 (WT) cell lines exhibit near-complete Ku80 knockout. Distinct Ku80 expression is observed at the GFP-Flag fusion site, confirming successful establishment of these cell lines. The GFP-Flag-Ku80 (K568R) and GFP-Flag-Ku80 (WT) cell lines are thus validated for use in subsequent examples.

In the following examples, all methods are conventional methods, unless otherwise specified.

The materials, reagents, and the like used in the following examples are all commercially available, unless otherwise specified.

### Example 1

Cells such as GFP-Flag-Ku80 (K568R) cell lines and GFP-Flag-Ku80 (WT) cell lines were prepared, treated with varying doses of ⁶⁰Co γ ray irradiation at a dose rate of 66.49 cGy/min, and harvested for subsequent analyses.

### (1) Colony Formation Assay

Cells exhibiting favorable growth status under microscopy were washed once with 1× PBS, trypsinized, centrifuged, and resuspended in complete medium to generate single-cell suspensions for counting. 6-well plates were prepared and labeled corresponding group names. Cell suspension volumes were calculated based on seeding densities specified in Table 1, with 3 repetitions per group:

TABLE 1
Cell inoculation numbers for colony formation assay

| | GFP-Flag-Ku80(WT) | GFP-Flag-Ku80(K568R) |
|---|---|---|
| 0 Gy | 1000 cells | 1000 cells |
| 1 Gy | 1000 cells | 1000 cells |
| 2 Gy | 1200 cells | 1200 cells |
| 4 Gy | 1600 cells | 1600 cells |
| 8 Gy | 2000 cells | 2000 cells |

After the inoculated cell attachment, the cells were irradiated with different ⁶⁰Co γ ray doses according to the groups.

After irradiation, 6-well plates were returned to the incubator or continued culture. During this period, the complete medium was replaced with fresh medium every 3 d. Cell growth was observed under a microscope until clones exceeded 50 cells/clone (approximately 14 d), medium in 6-well plates was discarded and cells were washed 1 time with pre-cold 1×PBS. 2 mL anhydrous methanol was added to each well for 1-h fixation. Methanol was removed and replaced with 2 mL Giemsa stain in each well for 3-h room temperature incubation.

After staining, Giemsa stain was recovered and 6-well plates were washed 3 times with water. The cells were then observed under a microscope and clones containing >50 cells were were selected and counted. Statistical analysis was performed using GraphPad Prism 9. The three independent experiments were conducted and data were expressed as mean±standard deviation (SD). * indicates P<0.1, ** indicates P<0.01, and *** indicates P<0.001.

As shown in FIG. 9, WT-Ku80 denoted GFP-Flag-Ku80 (WT) cell group; and K568R-Ku80 represented GFP-Flag-Ku80 (K568R) cell group. The results indicated that the Ku80 K568R mutation significantly affected clonogenicity capacity and clone size, significantly reduced cell survival rate. This Ku80 K568R mutation significantly affected both proliferative ability and radiation sensitivity.

(2) Cell Proliferation and Cell viability Assay

Cells exhibiting favorable growth status under microscopy were washed once with 1× PBS, trypsinized, centrifuged, and resuspended in complete medium to generate single-cell suspensions for counting. Meanwhile, an E-Plate was prepared by adding 50 μL complete medium per well and and placed in the multifunctional real-time label-free cell analyzer (RTCA) inside the cell culture incubator for blank background measurement with parameters set. Subsequently, 100 μL cell suspension containing 2,000 cells was added per well. Plates rested in a superclean bench for 30 min at room temperature before being transferred to the RTCA analyzer for culture, with 3 repetitions per group.

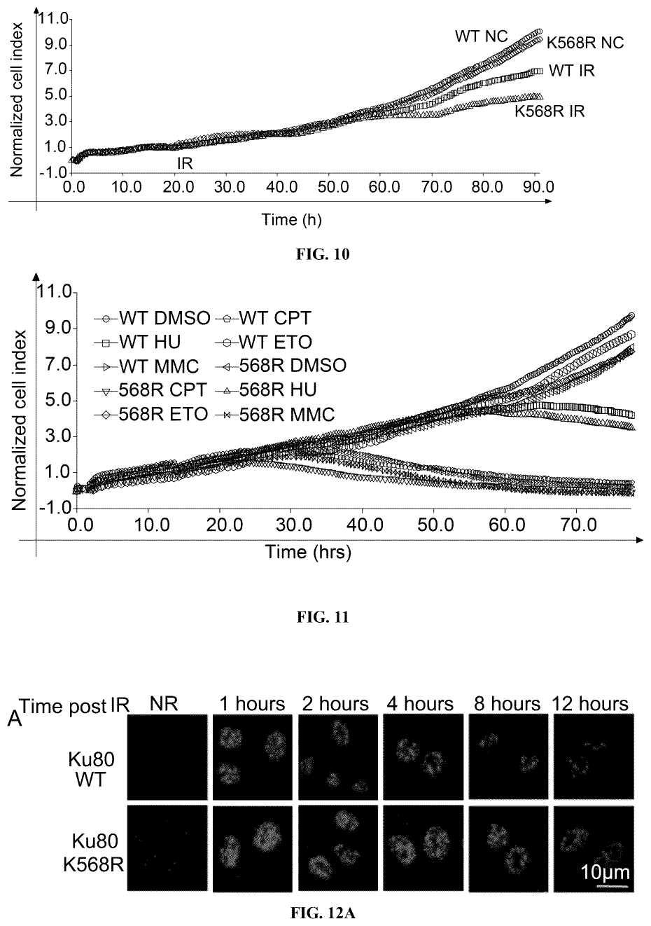

After cell attachment, E-Plate was taken out and irradiated with ⁶⁰Co γ ray (8 Gy). Real-time dynamic detection of cell proliferation was performed post-irradiation. Cells without ⁶⁰Co γ ray irradiation served as the control group. After the assay, the program was closed, data were saved, and the instrument was used for image processing and data analysis. As shown in FIG. 10, K568R NC represented non-⁶⁰Co γ ray-irradiated GFP-Flag-Ku80 (K568R) cell group; WT NC represented non-⁶⁰Co γ ray-irradiated GFP-Flag-Ku80 (WT) cell group; WT IR represented ⁶⁰Co γ ray-irradiated GFP-Flag-Ku80 (WT) cell group; and K568R IR represented ⁶⁰Co γ ray-irradiated GFP-Flag-Ku80 (K568R) cell group. FIG. 10 demonstrated that Ku80 K568R mutation significantly suppressed cell growth, demonstrating significant impacts on proliferative capacity and radiation sensitivity of cells by the Ku80 K568R mutation.

After cell attachment, E-Plate was taken out and medium in the wells was aspirated using a vacuum pump. Cells in wells were treated with CPT (camptothecin, 1 μM), HU (hydroxyurea, 1 mM), ETO (etoposide, 100 nM), and MMC (mitomycin C, 5 μM), respectively. Control wells received equivalent-volume DMSO. Real-time dynamic detection was continued. After the assay, the program was closed, data were saved, and the instrument was used for image processing and data analysis. Results (FIG. 11) showed that WT DMSO, WT CPT, WT HU, WT ETO, and WT MMC were GFP-Flag-Ku80 (WT) treated with DMSO, CPT, HU, ETO, and MMC; K568R DMSO, K568R CPT, K568R HU, K568R ETO, and K568R MMC were GFP-Flag-Ku80 (K568R) cell group treated with DMSO, CPT, HU, ETO, and MMC, respectively. The results show that compared to GFP-Flag-Ku80 (WT) cells, the viability of GFP-Flag-Ku80 (K568R) cells decreased significantly after treatment with these drugs, indicating that the Ku80 K568R mutation significantly affects cell proliferation ability and drug sensitivity.

### (3) Immunofluorescence Assay

Cover slips were pre-soaked in anhydrous ethanol and placed in 6-well plates prior to cell inoculation. Each well received 2 mL complete medium. Cells were uniformly inoculated in 6-well plates following standard passaging protocols and incubated in a cell culture incubator.

After cell attachment, cells were irradiated with varying doses of ⁶⁰Co γ ray. Cells were harvested at different time points post-irradiation (0 h, 1 h, 2 h, 4 h, 8 h, and 12 h) for processing: original medium in the 6-well plate was aspirated using a vacuum pump and cells washed 3 times with ice-cold 1× PBS. Each well was fixed with 2 mL 4% paraformaldehyde at room temperature for 30 min, then stored at 4° C. Processing of subsequent steps could be performed uniformly after fixation was completed for all time points.

After fixation, 4% paraformaldehyde in the 6-well plates was aspirated; wells were washed 2 times with 1× PBS, and 2 mL 0.3% Triton X-100 (room temperature, 20 min) was added to each well for permeabilization at temperature for 20 min. After permeabilization, the wells were washed 2 times with 1× PBS. 2 mL 3% BSA was added to each well for blocking at room temperature for 30 min. During blocking, primary antibody (γH2AX 1:200) was prepared in 3% BSA. Post-blocking, blocking solution was completely aspirated using a vacuum pump. Then 50 μL of the primary antibody was dropped onto the coverslip. And the plate was placed at 4° C. overnight or 2 h at room temperature for incubation. After primary incubation, 2 mL of ice-cold 1× PBS was added to each well, and the primary antibody was washed off using a shaker at medium speed; this was repeated three times. During this time, secondary antibody (goat anti-rabbit 1:400) was prepared in 3% BSA (light-protected). After washing the primary antibody, 50 μL secondary antibody applied was dropped onto the coverslip surface to allow light-protected incubation (room temperature, 1 h). Post-secondary incubation, 2 mL ice-cold 1× PBS was added to each well and the secondary antibody was washed using a shaker at high speed (light-protected), this was repeated three times. During washing of the secondary antibody, new glass slides were prepared, labeled with the corresponding sample names, and a drop of DAPI-containing anti-fade mounting medium was added to each slide. After washing, coverslips were carefully detached from 6-well plate and placed cell-side down onto the mounting medium on the glass slides, avoiding air bubbles as much as possible. Slides aluminum foil-wrapped and stored at 4° C. in humidified chambers.

After 1 d, images were acquired using confocal laser microscopy, followed by subsequent analysis and processing. Three independent replicate experiments were performed. Data are expressed as mean+SD; *** denotes P<0.001.

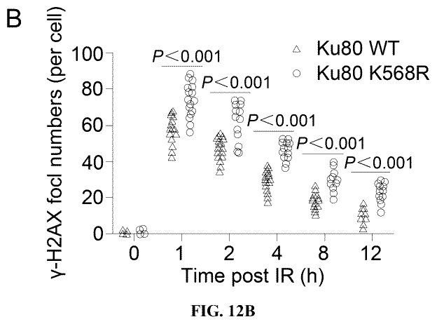

Results were shown in FIGS. 12A-12B. FIG. 12A: γ-H2AX foci detection by immunofluorescence in GFP-Flag-Ku80-WT and GFP-Flag-Ku80-K568R cells at different irradiation time; FIG. 12B: statistical graph of γ-H2AX foci. γ-H2AX serves as a DNA damage marker, and γ-H2AX undergoes phosphorylation to mark the damage sites to facilitate DNA damage repair in case of DNA damage. GFP-Flag-Ku80 (K568R) cells exhibited significantly increased γ-H2AX foci formation versus GFP-Flag-Ku80-WT cells, particularly at 8 h and 12 h post-irradiation. This indicated persistent DNA damage during late-phase after radiation and demonstrates significant impact on cell damage and inhibition of cellular repair by the Ku80 K568R mutation.

### (4) Flow Cytometric Analysis of Apoptosis

Cells exhibiting favorable growth status under microscopy were washed with 1×PBS, trypsinized, centrifuged, and inoculated in 12-well plates at 1×10⁴ cells/well. The plate was placed in the incubator for 12-h incubation.

Fresh complete medium was replenished prior to ⁶⁰Co γ ray irradiation. At 48 hours after 8 Gy irradiation, original medium in 12-well plate was transferred to 1.5 mL centrifuge tubes using a pipette. Cells were digested in 12-well plate per well with 300 μL trypsin and and the digestion was stopped using the corresponding original medium. Cell suspensions were transferred to 1.5 mL centrifuge tubes using a pipette and centrifuged at 4° C., 1,500 rpm for 3 min. After centrifugation, 1 mL ice-cold 1× PBS was added to each well. The cells were resuspended and washed by pipetting, then centrifuged again at 4° C., 1,500 rpm for 3 min. The above steps were repeated 2 times. After the last centrifugation, the supernatant was discarded, and the cells were gently resuspended in 100 μL 1× Annexin V Binding Buffer. Cell suspensions received 2.5 μL Annexin V-APC and 2.5 μL 7-AAD (light-protected), mixed gently using a pipette, and incubated at room temperature for 20 min. After incubation, 400 μL diluted 1× Annexin V Binding Buffer was added and gently mixed with a pipette. Samples were immediately analyzed by flow cytometry. Non-⁶⁰Co γ ray-irradiated cells served as controls.

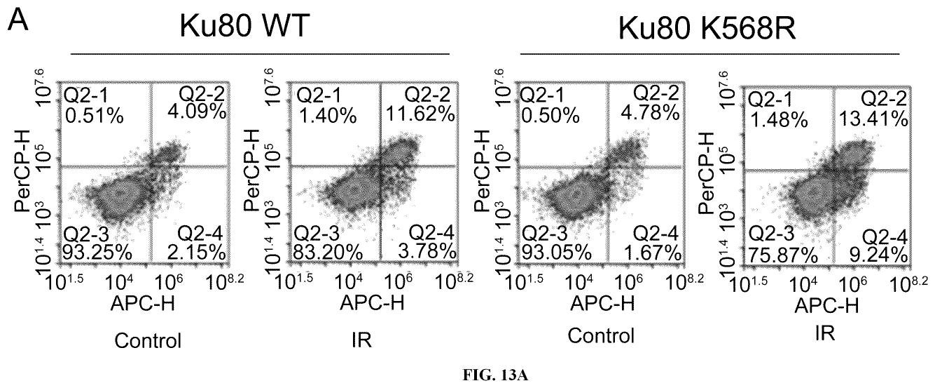

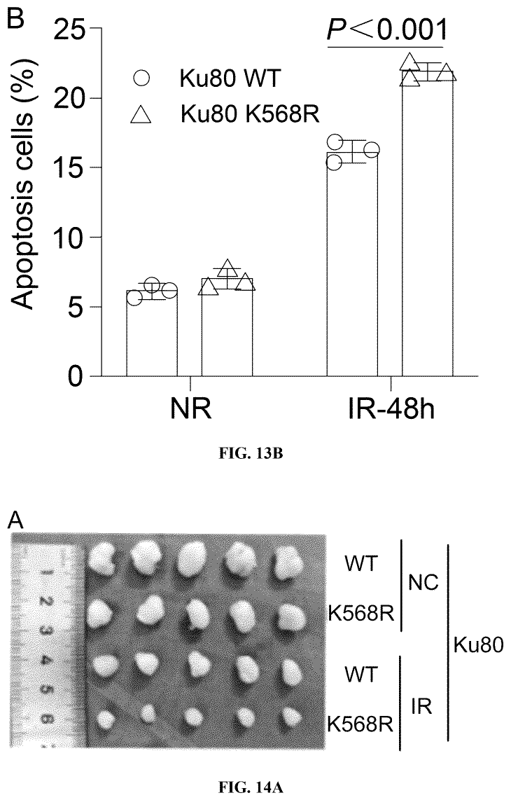

Results were shown in FIGS. 13A-13B. FIG. 13A showed apoptosis rate detected by flow cytometry; FIG. 13B is the statistical graph of the apoptosis rate, where NR represented non-irradiated and IR-48 h represented irradiation). Three independent replicate experiments were performed. Data were expressed as mean+SD; ns represented not significant; and ** represented P<0.01. GFP-Flag-(K568R) cells exhibited significantly increased apoptosis rate after irradiation versus GFP-Flag-(WT) cells, further demonstrating that the Ku80 K568R mutation increases cellular DNA damage.

### (5) Tumorigenicity Assay in Nude Mice

The experiment was divided into 4 groups: Ku80 WT NC group: injected with GFP-Flag-Ku80 (WT) cells (no ⁶⁰Co γ ray irradiation), Ku80 WT IR group: injected with GFP-Flag-Ku80 (WT) cells+⁶⁰Co γ ray irradiation, Ku80 K568R NC group: injected with GFP-Flag-Ku80 (K568R) cells (no ⁶⁰Co γ ray irradiation), and Ku80 K568R IR group: injected with GFP-Flag-Ku80 (K568R) cells+⁶⁰Co γ ray irradiation. 5 nude mice were in each group.

Prepared cells were microscopically examined for growth status and density. Cells exhibiting favorable conditions under microscope were trypsinized and centrifuged at 900 r/min for 3 min. Supernatant was discarded, followed by two washes with 1×PBS. After the last wash, partial cell suspension was taken for cell counting to adjust the concentration to 1×10⁶ cells per 100 μL. The cell suspension was maintained on ice for later use.

Nude mice from SPF (Specific Pathogen Free) environment were transferred to a superclean bench. Before inoculation, the cell suspension was dispersed by pipetting, A 1 mL syringe was used to draw 100 μL of the cell suspension, which was then subcutaneously injected into the mid-to-posterior region of the right axilla of the nude mice. During inoculation, the needle was inserted deeply (about 1 cm) under the skin and moved side-to-side several times after insertion to ensure accurate placement. Post-injection, needles were withdrawn slowly to prevent leakage of the cell suspension from the needle site. The inoculation should be slightly faster to ensure the activity of cells. Tumor growth was observed daily.

At 3 days post-injection, tumors became roughly visible to the naked eye, and localized 8 Gy ⁶⁰Co γ ray irradiation of the tumor area was administered. After irradiation, the longest and shortest diameters of the tumors were measured regularly with a caliper to calculate the relative tumor volume. Experiments terminated when tumors reached 1,000 mm³ to prevent excessive growth. The nude mice were cervically dislocated; tumors excised, tumor weighed, and photographed.

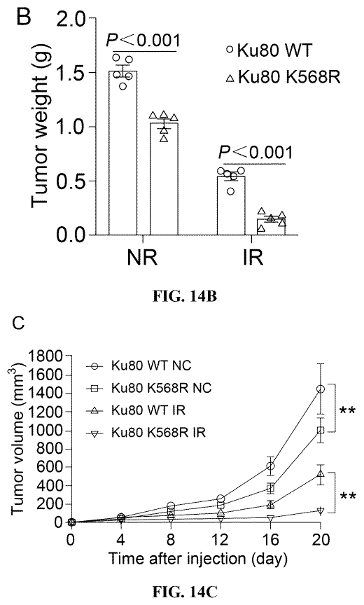

Results were shown in FIGS. 14A-14C. FIG. 14A shows photographs of tumors from each group; FIG. 14B: shows the tumor weight for each group; FIG. 14C shows the relative tumor volumes of each group. ** denoted P<0.01. The results show that the therapeutic effect of ionizing radiation treatment on tumors with Ku80 K568R mutation was significantly better than that of the GFP-Flag-(WT) cell group, manifested by significantly reduced tumor weight and volume in the Ku80 K568R mutant group. This indicated that the Ku80 K568R mutation inhibited tumor growth and promoted tumor radiosensitivity.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A method for inhibiting proliferation of a tumor cell, comprising: inducing a site-directed mutation at a K568 site of a Ku80 protein in the tumor cell.

2. The method according to claim 1, wherein the site-directed mutation at the K568 site is a substitution of K at position 568 of the Ku80 protein with R.

3. The method according to claim 1, wherein the tumor cell is a Ku80 protein-expressing tumor cell.

4. A method for inhibiting DNA damage repair in a tumor cell, comprising: inducing a site-directed mutation at a K568 site of a Ku80 protein in the tumor cell.

5. The method according to claim 4, wherein the site-directed mutation at the K568 site is a substitution of K at position 568 of the Ku80 protein with R.

6. The method according to claim 4, wherein the tumor cell is a Ku80 protein-expressing tumor cell.

7. A method for enhancing radiosensitivity of a tumor cell, comprising: inducing a site-directed mutation at a K568 site of a Ku80 protein in the tumor cell.

8. The method according to claim 7, wherein the site-directed mutation at the K568 site is a substitution of K at position 568 of the Ku80 protein with R.

9. The method according to claim 7, wherein the tumor cell is a Ku80 protein-expressing tumor cell.
